(19) United States
(12) Patent Application Publication
(10) Pub. No.: US 2009/0270900 A1
(43) Pub. Date: Oct. 29, 2009

(54) Multiple Purpose Body Pierce System

(76) Inventor: Edward Norman MacLeod, Westford, MA (US)

Correspondence Address: Edward MacLeod, 53 Pleasant Street, Westford, MA, 01886, US

(21) Appl. No.: 12/108,539

(22) Filed: Apr. 24, 2008

(51) Int. Cl. A61B 17/34

(52) U.S. Cl. 606/185

(57) ABSTRACT

A body friendly, multi-purpose, largely invisible, sub-dermal or trans-dermal body pierce system offering numerous applications. One embodiment is for the rapid mounting of extremely compact, attractive, comfortable, magnetically self aligning, temples, eyewear. Another is as a means for quick change, body friendly, pierce-style jewelry. Yet another is as a sub-dermal medication dispenser. The thin, flexible and essentially invisible pierce component is inserted with specialized tooling for minimal tissue damage and discomfort with a substantially reduced healing period.

### FIELD OF THE INVENTION

The field of the invention relates generally to body piercing methods and apparatuses. Specifically the present invention relates to multi-purpose body piercing methods and apparatuses that provide a means of eyewear mounting, a means of drug injection, a means of jewelry mounting, a means for housing micro-electronics.

### REFERENCES

In general within the art, descriptions of Frameless Glasses Attaching to Body in U.S. Pat. No. 7,066,592 set forth by Rose.

### BACKGROUND OF INVENTION

Various body pierce barbell style studs have been worn for years as methods of decoration and self-expression. These pierce studs were installed by first placing the stud into a tubular needle, inserting the needle through the skin, and then ejecting the stud as the needle is removed.

Some attempts have been made to add some additional functionality to these studs in order to provide an alternative means of eyewear. This provided some benefits over traditional eyewear; such as: the elimination of folding temples, eyewear frames and ear pieces. This also reduces size and weight, as well as the amount of contact between the eyewear correction devices and the wearer's skin surface, in addition to making storage more convenient and improving the wearer's comfort. Further, by removing the need for nose pads and having a secure means of attaching the vision correcting lens to the body pierce studs, eyewear would no longer slide down a wearer's nose and would no longer leave behind annoying nose pad marks.

However, these traditional barbell style studs, and attempts to add the ability to mount eyewear to these studs are lacking in many important aspects. For instance, traditional barbell style studs are comprised of rigid steel, which may damage underlying tissue and cause significant pain to wearers when the studs are impacted or snagged. Also, these studs use screw on ends that make it extremely difficult to adjust the metal stud length. Further, the removal of these studs is an awkward and cumbersome process. Still another drawback to these studs is that they should not be removed for at least several weeks after being inserted which makes it impossible for the wearer to change the stud for a different piece of jewelry. In addition, even after this initial healing period, to change jewelry, a wearer must change the actual stud they are currently wearing as well. Further, conventional stud ends inhibit peripheral vision when worn around the eye area.

The initial stud installation process too is lacking as the needle must have a significantly larger diameter than the stud, which leads to greater discomfort, pain and tissue damage when the needle is inserted into the skin.

Shortcomings involving attempts to mount eyewear to the body pierce studs involve all the flaws associated with traditional bar bell studs mentioned above, as these designs mount the eyewear directly to these traditional studs. These attempts attach separate lenses to a traditional barbell style stud, which would present practical difficulties. First, the two separate lenses would rarely be properly aligned and therefore would not provide proper vision correction, which over time could lead to additional vision loss.

Other shortcomings evident in the prior art related also exist. Specifically, these attempts utilize elastomeric clips to attach the eyewear to the traditional body pierce studs. In this design even a weak impact may overstress the pierce and cause injury and pain to the wearer. Further, the wearer must engage each elastomeric clip to mount the eyewear. This is an awkward, time-consuming multi-step operation that requires simultaneous use of both hands. To those that periodically remove their eyewear, by either requirement or personal choice, this is a chore that would be best to minimize.

Yet another drawback in prior attempts to mount eyewear onto these traditional barbell style studs centers around the excessive length of these studs, and they may be esthetically undesirable. Additionally, the lack of an easy length adjustment mechanism also becomes a notable shortcoming of the prior art.

In addition to the inadequacies illustrated in previous attempts to mount eyewear to barbell studs, there is a clear failure to implement additional functionality for the mount studs. Functions such as providing a means to insert drugs into the wearer's system for treatment, or allowing for the ability to remove jewelry without removing the stud to which it is mounted are also desirable.

What is needed is a stud like device that is non-intrusive, that can easily mount and demount eyewear, and or easily remove and mount pieces of jewelry that also affords wearers the ability to receive treatment drugs through the stud mount. The disclosed invention provides that functionality.

### SUMMARY OF THE INVENTION

The instant invention, as illustrated herein, is clearly not anticipated, rendered obvious, or even present in any of the prior art mechanisms, either alone or in any combination thereof. A minimally intrusive body piercing system, designed to overcome the previously mentioned shortcomings and also give users a much more effective means of installing a body piercing stud. Further, an apparatus designed to attach attachments of eyewear to the stud, jewelry to the stud and to allow for the insertion of treatment drugs through the body pierce stud.

Therefore, it is an object of the present invention to overcome the previously mentioned shortcomings found in traditional body piercing systems and pierced stud eyewear mount systems by providing a minimally intrusive body pierce system that causes minimal pain, discomfort and injury to the wearer when being inserted.

Another object of the present invention is to provide a minimally intrusive body pierce system that allows for the easy removal and mounting of eyewear.

Another object of the present invention is to provide a minimally intrusive body pierce system that allows for the easy removal of and mounting of conventional stud jewelry without the removal of the body pierce stud.

Another object of the present invention is to provide a minimally intrusive body pierce system that allows for conventional stud jewelry to be worn without making direct body contact, whereby less expensive non biocompatible stud materials to be used.

Another object of the present invention is to provide a minimally intrusive body pierce system that allows for the easy removal of and mounting of various accessories such as eyewear, jewelry and other components by utilizing a body pierce stud with magnetic endpoints.

Another object of the present invention is to provide a minimally intrusive body pierce system that provides a means for transmitting treatment drugs into a wearer's body.

Another object of the present invention is to provide a minimally intrusive body pierce system that is flexible.

Another object of the present invention is to provide a minimally intrusive body pierce system that possesses an easy-to-use length adjustment mechanism.

Another object of the present invention is to provide minimally intrusive body pierce system that is substantially unnoticeable to observers.

Another object of the present invention is to provide a minimally intrusive body pierce system that improves comfort and convenience for eyewear users.

Another object of the present invention is to provide a minimally intrusive body pierce system that is more lightweight and compact than traditional eyewear products such as eye glasses.

Another object of the present invention is to provide a minimally intrusive body pierce system that allows for a means of wearing frameless, templeless eyewear.

Another object of the present invention is to provide a minimally intrusive body pierce system that allows for eyewear removal of and mounting by the use of two or more fingers from the same hand in a short amount of time.

Another object of the present invention is to provide a minimally intrusive body pierce system that allows for eyewear that is up to 70% lighter than conventional eyewear.

Another object of the present invention is to provide a minimally intrusive body pierce system that allows for eyewear that is easier to clean than conventional eyewear having temple assemblies.

Another object of the present invention is to provide a minimally intrusive body pierce system that allows for eyewear that is mechanically more reliable than traditional eyewear having temple hinge assemblies.

Another object of the present invention is to provide a minimally intrusive body-pierce system that comprises approximately 15% of the gross volume of conventional eyewear (even when said conventional eyewear is folded).

Another object of the present invention is to provide a minimally intrusive body pierce system that allows for an eyewear system that provides unrestricted peripheral vision.

Another object of the present invention is to provide a minimally intrusive body-pierce system that comprises a hollow stud, wherein said hollow stud may house numerous elements.

Another object of the present invention is to provide a minimally intrusive body pierce system that comprises a hollow stud or a non-hollow stud, wherein said stud comprises magnetic plug ends.

Another object of the present invention is to provide a minimally intrusive body pierce system that comprises a hollow stud, wherein said stud serves as a protective mounting sleeve for conventional pierce style jewelry.

Another object of the present invention is to provide a minimally intrusive body pierce system that comprises a crafted surface stud, wherein said crafted surface of said crafted surface stud serves as a means increase the hold of the stud in the body tissue of the wearer.

Another object of the present invention is to provide a minimally intrusive body pierce system that comprises a hollow stud or a non-hollow stud, wherein said stud serves as housing for micro-electronic devices.

Another object of the present invention is to provide a minimally intrusive body pierce system that comprises a magnetic stud insertion system comprising insertion needles of substantially reduced diameters, wherein said needles cause substantially reduced damage to the body tissue of a wearer.

Another object of the present invention is to provide a minimally intrusive body pierce system that comprises a shouldered stud insertion system comprising insertion needles of substantially reduced diameters, wherein said needles cause substantially reduced damage to the bodily tissue of a wearer.

Another object of the present invention is to provide a minimally intrusive body pierce system that comprises a hollow stud or a non-hollow stud, wherein said stud serves as housing for Radio Frequency Identification (RFID) devices.

Another object of the present invention is to provide a minimally intrusive body pierce system that comprises a hollow stud or a non-hollow stud, wherein said stud serves as housing for Radio Frequency Intelligent tagging devices, wherein said devices communicate body sensor information such as temperature, blood glucose level, blood oxygen level.

Another object of the present invention is to provide a minimally intrusive body pierce system that comprises a clear walled stud, wherein said clear wall stud allows for the use of optoelectronic components within the stud.

Another object of the present invention is to provide a minimally intrusive body pierce system that comprises a hollow stud or a non-hollow stud, wherein said stud serves as housing for electronic sensor data recording devices, wherein said devices may upload data wirelessly or by direct electrical contact through the ends of the stud.

Another object of the present invention is to provide a minimally intrusive body pierce system that comprises a semi-permeable body pierce stud to dispense medication.

Another object of the present invention is to provide a minimally intrusive body pierce system that comprises a permeable body pierce stud to dispense medication.

Another object of the present invention is to provide a minimally intrusive body pierce system that comprises a hollow permeable body pierce stud to drain infection.

Another object of the present invention is to provide a minimally intrusive body pierce system that comprises stud, wherein the level of training necessary to implant/install said body pierce system is substantially less then the level of training required by that of a surgeon to implant electronic devices into a patient.

Another object of the present invention is to provide a minimally intrusive body pierce system that comprises an eyewear assembly, wherein said eyewear assembly further comprises nosepads and a magnetic nose bridge member.

Another object of the present invention is to provide a minimally intrusive body pierce system that comprises an eyewear assembly, wherein said eyewear assembly further comprises nosepads, a magnetic nose bridge member and a magnetic button to for storage of said eyewear on clothing.

Another object of the present invention is to provide a minimally intrusive body pierce system that comprises an eyewear assembly, wherein said eyewear assembly further comprises nosepads and a magnetic nose bridge member and allows the lenses of said eyewear assembly to fold substantially 90 degrees from their normal resting position substantially about a wearer's nose bridge.

Another object of the present invention is to provide a minimally intrusive body pierce system that comprises approximately only 15% of the bulk volume of conventional eyewear (even when said conventional eyewear is folded).

These together with other objects of the invention, along with various features of novelty which characterize the invention, are pointed out with particularity in the claims and Detailed Description of the Embodiments Sections, and drawings of this application, with all said sections also adding to this disclosure.

### BRIEF DESCRIPTION OF THE DRAWINGS

FIG. 1 illustrates side and front views of the eyewear lenses, the pierce, nose pads, an adjustable magnetic member and a magnetic stud assembly.

FIG. 2 illustrates side and front views of a substantially hidden magnetic stud assembly without the other eyewear members.

FIG. 3 illustrates a substantially hidden stud with magnetic ends, a bio-compatible stud sleeve and a magnetic plug.

FIG. 4 illustrates a partial cross sectional view of several stud components including stud sleeves, end plugs, a quick release press fit and a magnet.

FIG. 5 illustrates a substantially hidden stud with magnetic ends with a bio-compatible stud sleeve and an example of a crafted surface retaining feature, in this case a raised helical thread.

FIG. 6 illustrates magnetic eyewear storage features of the instant inventions. In this figure the instant invention is held against clothing by a magnetic pair and a magnetically attached item.

FIG. 7 illustrates a stud with magnetic ends comprising a bio-compatible stud sleeve, a magnetic plug, a magnetic end and a flange.

FIG. 8 illustrates a stud installation tool compatible with installing studs suitable for the instant invention. This installation tool comprises a partial stud assembly, a piercing needle, a magnetic tool stud interface.

FIG. 9 illustrates side and front views of the eyewear lenses, the pierce, nose pads with molded in magnetic member and a magnetic stud assembly.

FIG. 10 illustrates a front view of the eyewear lenses, nose pads, an adjustable magnetic member and a magnetic stud assembly, wherein said eyewear lenses may be folded upward to substantially a 90 degree angle from the normal lenses eyewear viewing position.

FIG. 11 illustrates the body-pierce system installation tool for use in the shoulder alignment body-pierce insertion technique. In addition it shows the hollow stud as used as a protective sleeve for conventional stud style jewelry.

FIG. 12 illustrates a side view of the stud assembly, wherein said stud assembly contains a micro-electronics or chemical sensor payload.

### DETAILED DESCRIPTION OF THE DISCLOSED EMBODIMENTS

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention, such as body-pierce devices comprised of different materials and/or combinations of materials as well as body-pierce devices of various dimensions and sizes.

Presently, there are no body-pierce devices that serve all the functions of the instant invention. This system provides a convenient and comfortable means of eyewear, a mount for a wearer to easily add and remove jewelry, and also serves as a conduit through which medication or fluid may pass. Previous body-pierce systems fail to allow for all these applications and fail to perform even the eyewear function with the convenience that the instant invention provides.

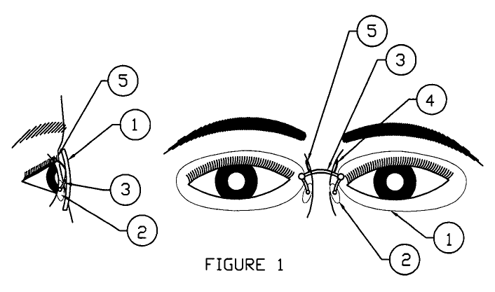

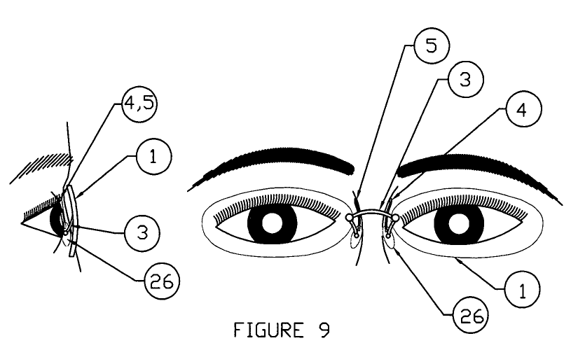

FIGS. 1 and 9 illustrate several common components of the invention through a front and side view of the invention, respectively when mounted in front of a person's face. To this said face front is mounted the front section of a typical piece of eyewear including the lenses 1 and nose-pads 2 (but excluding temples and temples mounting assemblies). Also illustrated, and, unique to this invention, are a magnetic nose-pad-stud-bridge assembly 3, an adjustable magnetic yoke lens assembly 4, and a magnetic stud assembly 5 hereafter referred to as stud assembly. FIG. 9 alone further depicts 26 nosepads with integral magnetic elements 4.

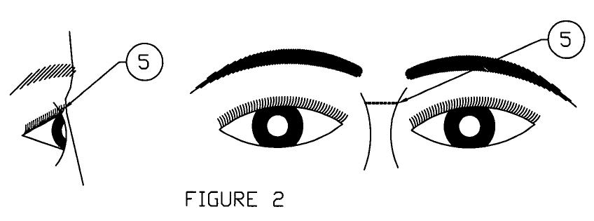

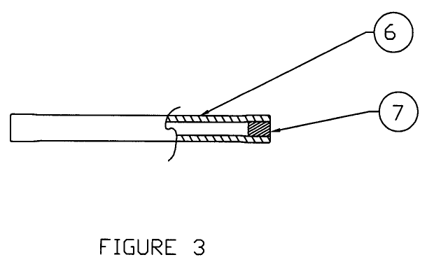

FIGS. 2, 3 illustrate the substantially hidden stud 5 and its various components. FIG. 2 specifically illustrates a front and side view of the substantially hidden stud 5 when worn within the soft tissue of the bridge of a wearer's nose.

FIG. 3 illustrates an internal and external view of the substantially hidden stud within a bio-compatible stud sleeve 6 and a magnetic plug 7 at one end of said stud 5, wherein said stud sleeve 6 bulges outwardly substantially about the area where said magnetic plug 7 lies within said stud sleeve 6. Said bulge is just one example of crafting the exterior surface of the stud sleeve to improve hold within the body.

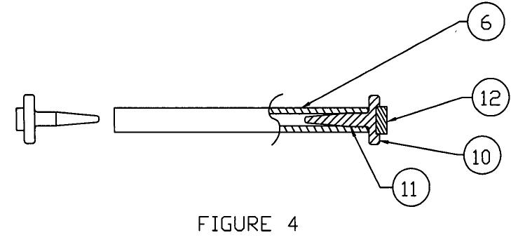

FIG. 4 illustrates an internal and external view of the stud detailing a quick release tapered plug 11 with flange 10, and an optional magnet member 12 affixed to the end of said tapered plug 11.

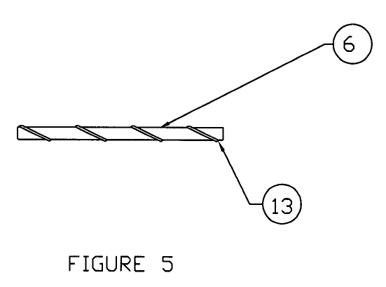

FIG. 5 illustrates an external view of the stud sleeve 6 and an example retaining feature 13. Said example retaining feature 13 is a substantially helical pattern about said sleeve 6 or being comprised of material protruding out of said sleeve 6 in a substantially helical pattern. Additional obvious and possible retaining features include but are not limited to dimples, bulges, shoulders and surface roughness. Retaining feature 13 may protrude above or be impressed within the nominal surface of stud sleeve 6.

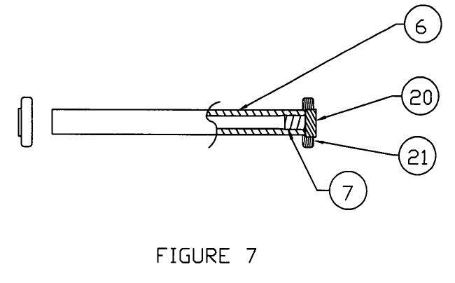

FIG. 7 also illustrates an internal and external view of said stud 5, disclosing the sleeve 6, the magnetic plug 7, a magnetic end member 20 and a flange 21.

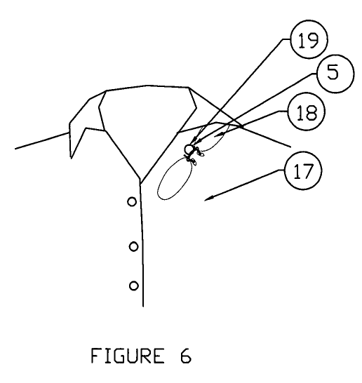

FIG. 6 illustrates a quick storage eye-wear carrying system comprising the eyewear device with magnetic elements 18 held in place to clothing 17 with the use of a magnetic pair 19 between which is the clothing to which said eyewear device attachedly rests.

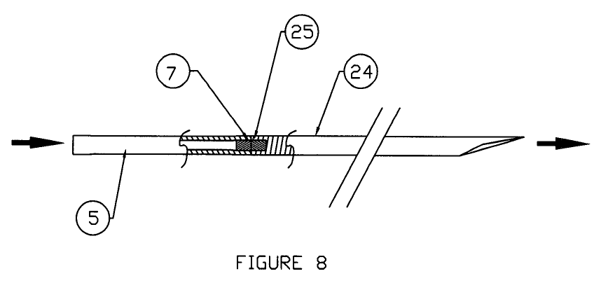

FIG. 8 illustrates a magnetic stud installation tool 24 in which an end of the stud assembly 5 is magnetically affixed to the end of body-pierce needle 23. Further illustrated in this figure is a needle-stud interface 25 that is used to pull the stud assembly 3 into place. As the diameter of needle 25 is approximately the same diameter is the stud 5 there is significantly less user tissue damage than caused by the prior art.

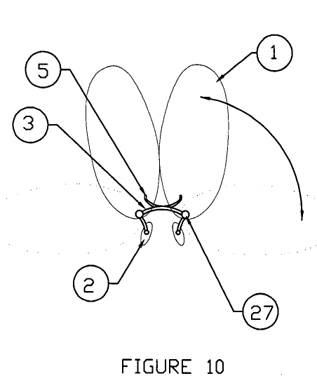

FIG. 10 illustrates how the lenses 1 may be flipped up as well as rotated in such a way as to remove them from the line of sight from the wearer, without the wearer having to remove the eyewear completely. This upward rotation is accomplished by way of at least one pivot member 27, attached to the bridge assembly 3.

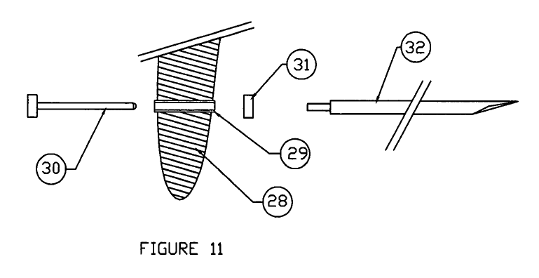

FIG. 11 illustrates the installation needle 32 which removably mates with and positions said stud sleeve 6 in the body using an alignment post 29. The needle 32 and stud 6 are inserted in the body (herein shown as an earlobe 28) as an assembly. The needle is pulled away or otherwise disengaged when correct positioning of the stud sleeve has been made within the body.

FIG. 11 additionally illustrates the use of stud sleeve 6 as a protective liner for a conventional body pierce whereby the conventional stud jewelry 30, with stud back 31 shown for reference, may be easily inserted into or removed from the sleeve 6 without disrupting or making direct contact with the body tissue 28.

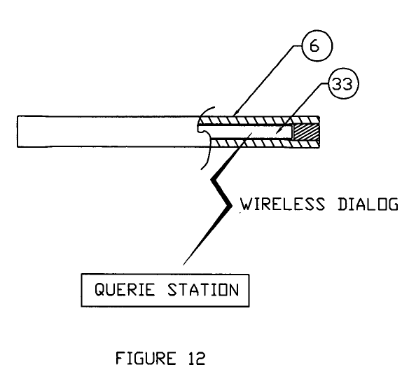

FIG. 12 illustrates a side view of the stud assembly, wherein said stud assembly contains a microelectronics or chemical sensor payload. Numerous useful payloads, apparent to those skilled in the art, would benefit from being positioned within the body within the stud sleeve 6.

In other embodiments of this invention the dimensions, shapes and construction materials of the multi-function body pierce eyewear apparatus may vary including the dimensions, shapes and construction materials of the stud, stud installation tool and components used in the magnetic eyewear storage device.

What is claimed is:

1. A body-pierce system comprising a stud wherein said stud comprises a hollow interior.

2. The body-pierce system of claim 1, wherein said stud is non metallic and may be cut to length with a sharp edge.

3. The body-pierce system of claim 1, wherein said stud is fitted with at least one removable tapered plug end.

4. The body-A body-pierce system of claim 1 wherein an exterior of said stud is substantially crafted to control hold between the stud and body tissue.

5. The body-pierce system of claim 1, wherein said stud is fitted with at least one magnetic plug end.

6. The body-pierce system of claim 1, wherein said stud is fitted with two magnet plug ends comprising substantially similar poles disposed to face each other.

7. The body-pierce system of claim 1 wherein a wall of said stud is substantially permeable.

8. The body-pierce system of claim 7 wherein said stud contains medication and said medication is dispensed under the skin.

9. The body-pierce system of claim 1 wherein said stud contains at least one electronic device.

10. The body-pierce system of claim 1 wherein said stud contains at least one sensor.

11. The body-pierce system of claim 1 further comprising a body pierce insertion tool for said stud comprising a needle of a substantially similar outside diameter with that of said stud and having a male alignment pin sized for a slip fit with the inside diameter of said stud.

12. The body-pierce system of claim 5 further comprising a body pierce insertion tool for said stud comprising a needle of similar outside diameter as that of said stud and having a magnet end of appropriate polarity and strength to help pull said stud into position in the body.

13. The body-pierce system of claim 5 further comprising an eyewear mounting system comprising a conventional nosepad, lens and eyewear bridge structure to which is fitted at least one magnetic element disposed to magnetically and removably mate with said user worn stud assembly.

14. The body-pierce system of claim 6 further comprising an eyewear mounting system comprising a conventional nosepad, lens and eyewear bridge structure to which is fitted at least one magnetic element disposed to magnetically and removably mate with said user worn stud assembly.

15. The body-pierce system of claim 14 wherein said stud assembly is worn horizontally and disposed within a nose bridge of a user.

16. The body-pierce system of claim 13 wherein said eyewear is fitted with at least one positionable magnet, which when worn, mates with the magnetic pole of the stud assembly.

17. The body-pierce system of claim 13 wherein the eyewear mounting system is fitted with two positionable magnets which magnetically mate with the magnetic ends of the stud assembly of claim 6.

18. The body-pierce system of claim 13 wherein a set of lenses of said eyewear mounting system may be individually pivoted upward within the facial plane to a position that is substantially out of line of sight of the user.

19. The body-pierce system of claim 13 wherein said set of lenses of said eyewear mounting system may be simultaneously pivoted outward and upward about the horizontal axis, to a position that is substantially out of line of sight of the user.
